(19) United States
(12) Patent Application Publication
(10) Pub. No.: US 2023/0135521 A1
(43) Pub. Date: May 4, 2023

(54) PHARMACEUTICAL FORMULATIONS FOR NOVEL FELINE ERYTHROPOIETIN RECEPTOR AGONISTS

(71) Applicant: AskGene Pharma Inc., Camarillo, CA (US)

(72) Inventors: Yuefeng Lu, Newbury Park, CA (US); Jian-Feng Lu, Oak Park, CA (US); Lu Li, Camarillo, CA (US); Ming Li, Thousand Oaks, CA (US)

(73) Assignee: AskGene Pharma Inc., Camarillo, CA (US)

(21) Appl. No.: 17/049,915

(22) PCT Filed: Apr. 22, 2019
(86) PCT No.: PCT/US2019/028514

§ 371 Date: Oct. 22, 2020

Related U.S. Application Data
(60) Provisional application No. 62/661,045, filed on Apr. 22, 2018.

(51) Int. Cl. A61K 38/18; A61K 9/08; A61K 47/26; A61K 47/02; A61K 47/18

(52) U.S. Cl. CPC A61K 38/1816; A61K 9/08; A61K 47/26; A61K 47/02; A61K 47/183

(57) ABSTRACT

The present specification discloses a pharmaceutical composition or stable aqueous formulations for erythropoietin receptor agonists, compositions and medicaments comprising such erythropoietin receptor agonists, and methods and uses of erythropoietin receptor agonists and compositions. The present specification further discloses medicaments for treating an anemia in non-human mammals such as cats.

### CROSS REFERENCE TO RELATED APPLICATIONS

The present application is a national stage application of PCT/US19/28514 and claims priority from U.S. Provisional Application 62/661,045, filed Apr. 22, 2018. The contents of the aforementioned priority applications are incorporated herein by reference in their entirety.

### INTRODUCTION

Non-regenerative anemia (NRA) is a serious disease in cats, dogs and other mammals and there are no adequate therapies. Erythropoietin (EPO) is a glycoprotein hormone and is the most important hormone regulating erythropoiesis (red blood cell production). Recombinant human EPO products such as the non-glycosylated EPOGEN® and the glycosylated ARANESP® (darbepoetin) have been commercially available to treat NRA in humans. Human EPO is a heavily glycosylated protein with a molecular mass of 30.4 kD. The 5 exons of the EPO gene encode 193 amino acids, 27 of which are later cleaved off to produce a 166-amino acid long peptide, although the circulating peptide contains 166 amino acids. Its mature, circulating, structure includes a 166-amino acid backbone with three N-linked carbohydrates attached to asparagine residues at amino acid positions 24, 38, and 83 and one O-linked carbohydrate attached to Ser¹²⁶. Sixty percent of the EPO molecular mass is contributed by the 166 amino acids and 40% is contributed by the carbohydrate. The carbohydrates probably cover much of the surface of the molecule since they have extended and flexible structure.

The carbohydrate residues allow for many possible isoforms and contribute significantly to the serum half-life and biological activity of the hormone in vivo. The isoforms with increased sialic acid content have longer serum half-life and reduced receptor binding affinity. For EPO, the serum half-life is the primary determinant of in vivo activity. Darbepoetin was created through site directed mutation of two amino acid residues, allowing for two additional N-linked carbohydrate chains.

It is estimated that there are over 0.5-1 million domestic cats (“cats”) that are suffering from NRA in the United States. Based on a veterinary survey, a number of conditions are associated with and/or can lead to NRA in cats. These include chronic renal failure (51%), cancer (16%), retroviral disease (11%), hyperthyroidism (5%), inflammatory bowel disease (4%), as well as other miscellaneous chronic conditions (6%) or multiple miscellaneous conditions (6%). Similar to the incidence rate in humans, chronic renal diseases and cancers are two main causes of NRA in cats.

There is currently no adequate treatment for NRA in cats. Internal surveys have indicated that currently available treatments for NRA include EPOGEN® or other human recombinant EPO (39%), ARANESP® (5.7%), corticosteroids (32.5%), blood transfusions (6.7%), anabolic steroids (1.4%), as well as other miscellaneous treatments (5.3%). However, antibodies often develop in some of the cats against those human proteins, which resulted in serious conditions. These include pure red-cell aplasia (PRCA) in 25-30% of cats. ARANESP® may have lower occurrence of PRCA in cats, estimated to be less than 10%, but it is still significant. The second most frequently used option is steroids, which often result in serious side effects and dubious therapeutic efficacy. Blood transfusion is inconvenient and expensive. Thus, there is currently a serious unmet veterinary need for cats with NRA.

No species-specific EPO is commercially available for cats as of today. Feline EPO is only about 83% similar to that of the human EPO. Recombinant feline EPO was produced and tested in cats. Unfortunately, development of red cell aplasia was observed at a similar rate as that of recombinant human EPO. Thus, unmodified recombinant feline EPO will likely not address this unmet veterinary need.

Peptide-based erythropoietin receptor (EpoR) agonists have also been developed, such as those disclosed in U.S. Pat. No. 6,703,480. In a small clinical trial, a peptide-based EpoR agonist was also shown to be effective for red cell aplasia in humans. It is believed that the peptide-based EpoR agonist would not induce red cell aplasia as it does not share any sequence similarities to that of EPO. However, no studies have been reported using the peptide-based EpoR agonist in veterinary applications. In addition, hypersensitivity was also reported in a small number of patients using the pegylated peptide-based EpoR.

### SUMMARY OF THE INVENTION

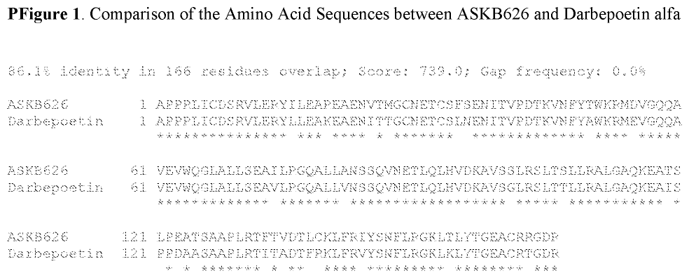

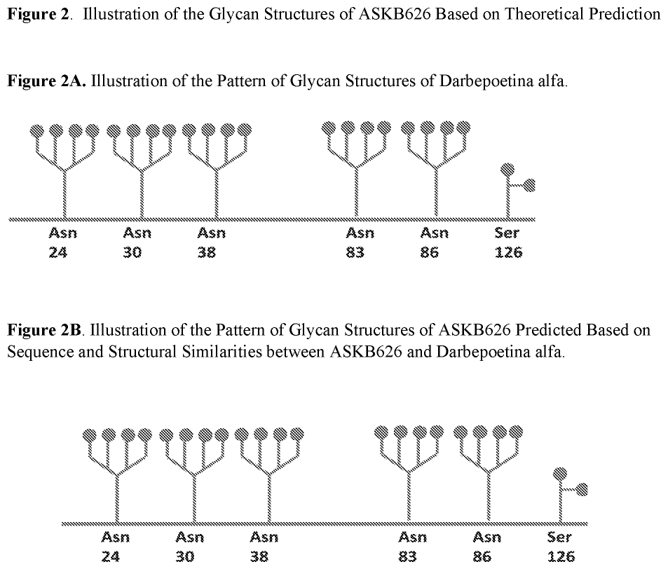

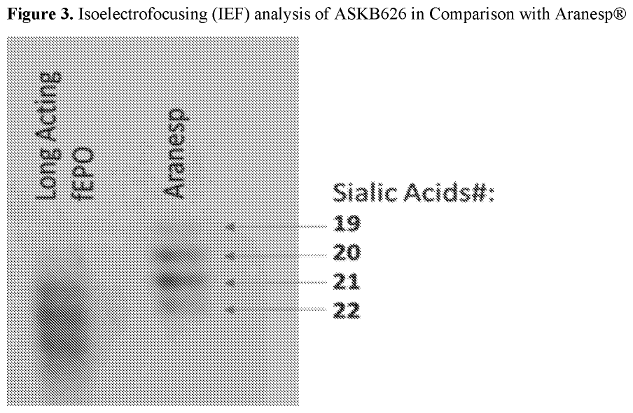

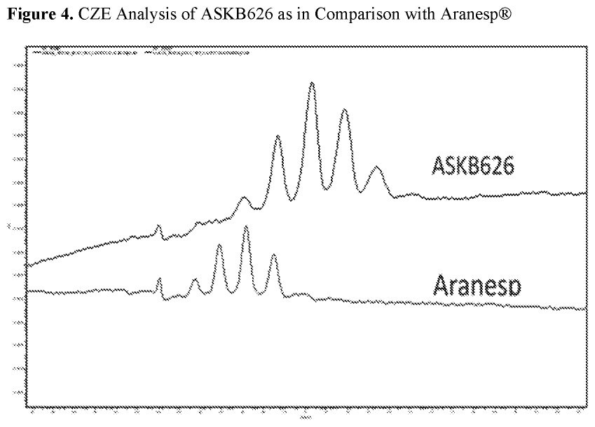

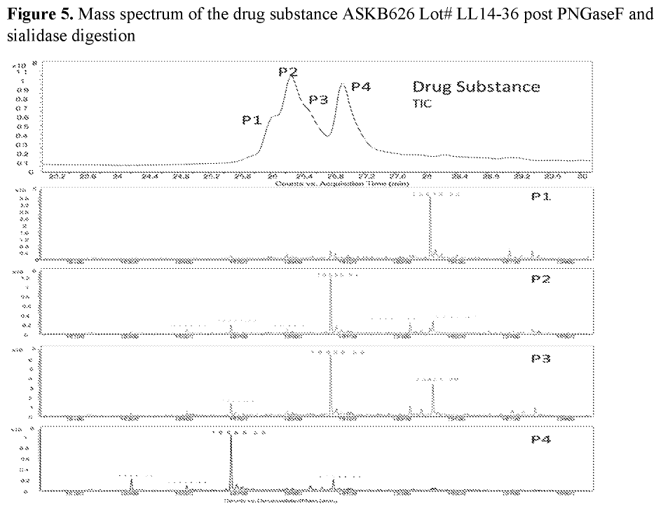

A novel long acting recombinant feline erythropoietin (EPO) analog was disclosed in patent application U.S. Ser. No. 14/857,799, herein incorporated by reference in its entirety. This molecule has since been moved into clinical development and with a product name ASKB626. ASKB626 has approximately 86% sequence homology to Aranesp (Darbepoetina alfa), a long lasting recombinant human EPO analog (FIG. 1). Because of the sequence and structure similarity, one would expect that ASKB626 would have glycan patterns that are similar to that of Aranesp, as illustrated in FIG. 2. However, their turns out that recombinant feline EPO analog molecules in the ASKB626 pharmaceutical preparation were on average significantly more negatively charged than Aranesp as analyzed by IEF (FIG. 3) and capillary zone electrophoresis (CZE) (FIG. 4). This more negative charge is found even though it only contains one less positive charge when compared to Aranesp. Further analysis led to the surprising discovery that the ASKB626 pharmaceutical preparations comprised a significant percentage of molecules with two O-linked glycans (FIG. 5) in addition to five N-glycans (Table 2), as comparing to Aranesp or the theoretical illustration of the glycan structure of ASKB626 (FIG. 1). Because of the importance of glycosylation in the biological activities and therapeutic efficacy of erythropoietin-based therapeutics, in an embodiment we developed novel pharmaceutical compositions with even higher levels of glycosylation than expected.

In addition to the differences in the patterns of glycan structures, it was also observed that Darbepoetina alfa had significantly higher in vitro cell-based activities than that of ASKB626 in the TF-1 proliferation assay and TF-1 functional fEPO assays (data not shown). A similar difference was reported between human EPO and feline EPO in a similar TF-1 type assay, where the human EPO was approximately 6 times more potent than feline EPO based on the values of EC50 (U.S. Pat. No. 9,644,014 B2).

It is clear that a difference of approximately 14% in the amino acid sequences of ASKB626 and Darbepoetina alfa can lead to significant differences in structural and biochemical properties and potentially in vitro and in vivo efficacy and safety profiles.

The present specification discloses pharmaceutical preparations or stable sterile aquous formulation for the recombinant feline erythropoietin (EPO) analog (SEQ ID NOS:1-6). The present specification also discloses pharmaceutical compositions or a stable sterile aqueous formulation of a recombinant feline EPO analog with enhanced in vivo half-life and reduced immunogenicity.

In some aspect, a pharmaceutical composition or a stable sterile aqueous formulation used to treat anemia in cats or dogs with a recombinant feline EPO analog molecule that has an amino acid sequence at least 95% identical to SEQ ID NO: 1; and wherein at least 95% of said EPO analog molecules comprise five (5) N-glycans per molecule, at least 85% of said EPO analog molecules comprise at least one (1) O-glycans per molecule, and at least 10% of said EPO analog molecules comprise at least two (2) O-glycans per molecule.

In some embodiments, a recombinant feline EPO analog comprises an amino acid sequence selected from SEQ ID NOS: 1-6.

In some embodiments, a pharmaceutical composition or stable aqueous sterile formulation comprises a protein concentration of 0.005-0.10 mg/ml, and a polysorbate-20 or polysorbate-80 concentration of 0.02-0.2 mg/ml; and wherein the pharmaceutical composition or stable aqueous sterile formulation has a pH of 5.0-6.5.

In some embodiments, a pharmaceutical composition or a stable aqueous sterile formulation comprises a protein concentration of 0.01-0.05 mg/ml, a polysorbate-80 concentration of 0.025-0.1 mg/ml, a phosphate concentration of 15-25 mM and sodium chloride at a concentration of 5-10 mg/ml; and wherein the formulation has a pH of 5.9-6.5.

In some embodiments, a pharmaceutical composition or a stable aqueous sterile formulation comprises a protein concentration of approximately 0.025 mg/ml, a polysorbate-80 concentration of approximately 0.05mg/ml, a sodium phosphate monobasic monohydrate of approximately 2.12 mg/ml, a sodium phosphate dibasic anhydrous at a concentration of approximately 0.66 mg/ml, and sodium chloride at a concentration of approximately 8.18 mg/ml; and wherein the pharmaceutical composition or stable aqueous sterile formulation has a pH at 6.2±0.2.

In some embodiments, a pharmaceutical composition or a stable aqueous sterile formulation comprises a protein concentration of approximately 0.025 mg/ml, a polysorbate-80 concentration of approximately 0.05 mg/ml, an acetate concentration of 10-20 mM, and a sodium chloride concentration at 5-10 mg/ml; and wherein the pharmaceutical composition or stable aqueous sterile formulation has a pH of 5.0-5.8.

In some embodiments, a recombinant feline EPO analog molecules in the pharmaceutical composition or stable aqueous sterile formulation has on average a greater negative charge than Darbepoetin alpha, and wherein at least one of the three most abundant band of the recombinant EPO analog molecule runs lower than the lowest band of the four major bands of Darbepoetin alpha as analyzed IEF analysis.

In some embodiments, a recombinant feline EPO analog molecule in the pharmaceutical composition or stable aqueous sterile formulation has on average a greater negative charge than Darbepoetin alpha, and wherein none of the three most abundant bands of the recombinant feline EPO analog runs higher than the second highest band of the four major bands of Darbepoetin alpha as analyzed by IEF analysis.

In some embodiments, a recombinant feline EPO analog molecules in the pharmaceutical composition or stable aqueous sterile formulation has on average have a greater negative change than Darbepoetin alpha, and wherein none of the three most abundant peaks of the recombinant feline EPO analog eluted earlier than the second earliest eluted peak of the three most abundant peaks of Darbepoetin alpha as analyzed by CZE analysis.

In some embodiments, recombinant feline EPO analog molecules in the pharmaceutical composition or stable aqueous sterile formulation have more negative charges than Darbepoetin alpha, wherein at least two of the three most abundant peaks of the said recombinant feline EPO analog eluted later than the last eluted peak of the three most abundant peaks of Darbepoetin alpha as analyzed by CZE analysis.

In some embodiments, said recombinant feline EPO analog in the said pharmaceutical composition or stable aqueous sterile formulation is glycosylated; wherein at least 95% of the recombinant feline EPO analog molecules contain five N-glycans per molecule, at least 85% of said EPO analog molecules comprise at least one (1) O-glycans per molecule, and at least 20% of the feline EPO analog molecules contain two or more O-linked glycans per molecule.

In some embodiments, a recombinant feline EPO analog in the said pharmaceutical composition or stable aqueous sterile formulation is glycosylated; wherein at least 95% of the recombinant feline EPO analog molecules contain five N-glycans per molecule, at least 85% of said EPO analog molecules comprise at least one (1) O-glycans per molecule, and at least 30% of the feline EPO analog molecules contain two or more O-linked glycans per molecule.

In some embodiments, a recombinant feline EPO analog in the said pharmaceutical composition or stable aqueous sterile formulation is glycosylated; wherein at least 95% of the recombinant feline EPO analog molecules contain five N-glycans per molecule, at least 85% of said EPO analog molecules comprise at one (1) O-glycans per molecule, at least 85% of said EPO analog molecules comprise at least one (1) O-glycans per molecule, and at least 40% of the feline EPO analog molecules contain two or more O-linked glycans per molecule.

In some embodiments, a recombinant feline EPO analog in the said pharmaceutical composition or stable aqueous sterile formulation is glycosylated; wherein at least 95% of the feline EPO analog polypeptide molecules in said formulation comprise a total of 18 or more sialic acids in the glycans of each said EPO analog molecule.

In some embodiments, a recombinant feline EPO analog in the said pharmaceutical composition or stable aqueous sterile formulation is glycosylated; wherein at least 95% of the feline EPO analog molecules in said formulation comprise a total of 19 or more sialic acids in its glycans of each said EPO analog molecule.

In some embodiments, a recombinant feline EPO analog in the said pharmaceutical composition or stable aqueous sterile formulation is glycosylated; wherein at least 98% of the feline EPO analog polypeptide molecules in said formulation comprise a total of 19 or more sialic acids in its glycans of each said EPO analog molecule.

In some embodiments, a recombinant feline EPO analog in the said pharmaceutical composition or stable aqueous sterile formulation is glycosylated; wherein at least 90% of the feline EPO analog molecules in said formulation comprise a total of 20 or more sialic acids in its glycans of each said EPO analog molecule.

In some embodiments, said pharmaceutical composition or stable aqueous sterile formulation comprises mostly monomeric recombinant feline EPO analog; wherein the level of aggregated protein is less than 2% as analyzed by a SEC-HPLC method.

In some embodiments, said pharmaceutical composition or stable aqueous sterile formulation comprises further comprises 2-20 mM methionine.

In some embodiments, said pharmaceutical composition or stable aqueous sterile formulation comprises further comprises sucrose or trehalose.

Also disclosed is a method for treating anemia in a subject, wherein said subject comprise a cat or a dog, said method comprising administering to a subject in need of such a treatment any of above said pharmaceutical composition.

In some embodiments, said subject is a cat; and wherein the amount of the feline EPO analog dosed is 0.0025-0.020 mg per week per cat. In some embodiment, the amount dosed is 0.001-0.004 mg/kg of body weight per week per cat or dog. In some embodiment, the amount dosed is 0.001-0.004 mg/kg of body weight per two weeks per cat or dog. In some embodiment, said subject is a cat; and wherein the amount dosed is 0.001-0.002 mg/kg of bodyweight per week per cat. In some embodiment, said subject is a cat; and wherein the amount dosed is approximately 0.001 mg/kg of body weight per week per cat. In some embodiment, said subject is a cat; and wherein the amount dosed is approximately 0.0015 mg/kg of body weight per week per cat. In some embodiment, said subject is a cat; and wherein the amount dosed is approximately 0.002 mg/kg of body weight per week per cat.

In one aspect, the stable aqueous sterile formulation for multidose injection is in a single vial.

In one aspect, the invention provides a method for treating anemia in a subject, wherein said subject comprises a cat or a dog, said method comprising administering to a subject in need of such a treatment a pharmaceutical composition as described above.

BRIEF DESCRIPTIONS OF THE DRAWINGS

FIG. 1. Comparison of the Amino Acid Sequences between ASKB626 and Darbepoetin alfa

FIG. 2. Illustration of the Glycan Structures of ASKB626 Based on Theoretical Prediction. FIG. 2A. Illustration of the Pattern of Glycan Structures of Darbepoetin alfa. FIG. 2B. Illustration of the Pattern of Glycan Structures of ASKB626 Predicted Based on Sequence and Structural Similarities between ASKB626 and Darbepoetin alfa.

FIG. 3. Isoelectrofocusing (IEF) analysis of ASKB626 in Comparison with Aranesp®

FIG. 4. CZE Analysis of ASKB626 as in Comparison with Aranesp®.

FIG. 5. Mass spectrum of the drug substance ASKB626 Lot#LL14-36 post PNGaseF and sialidase digestion

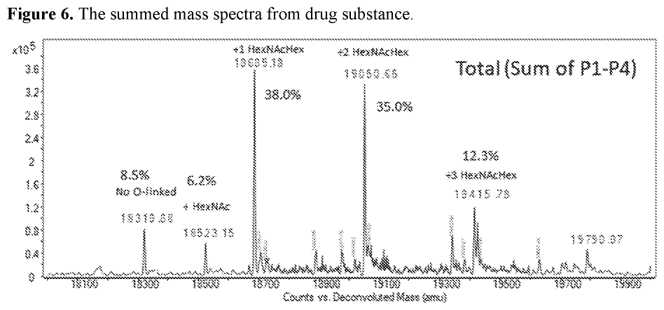

FIG. 6. The summed mass spectra from drug substance ASKB626 Lot#LL14-36 post PNGaseF and sialidase digestion.

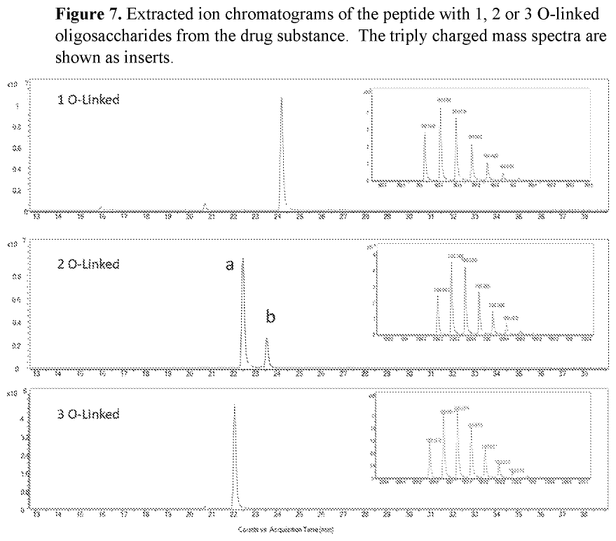

FIG. 7. Extracted ion chromatograms of the peptide with 1, 2 or 3 O-linked oligosaccharides from the drug substance. The triply charged mass spectra are shown as inserts.

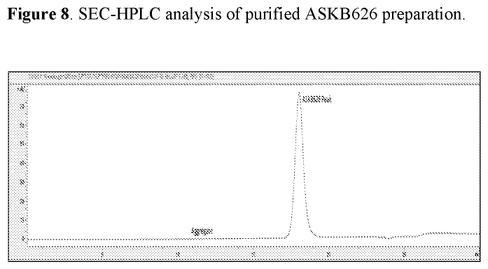

FIG. 8. SEC-HPLC analysis of purified ASKB626 preparation.

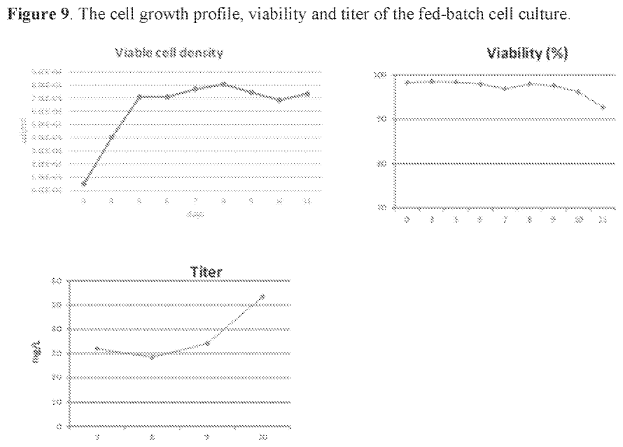

FIG. 9. The cell growth profile, viability and titer of the fed-batch cell culture.

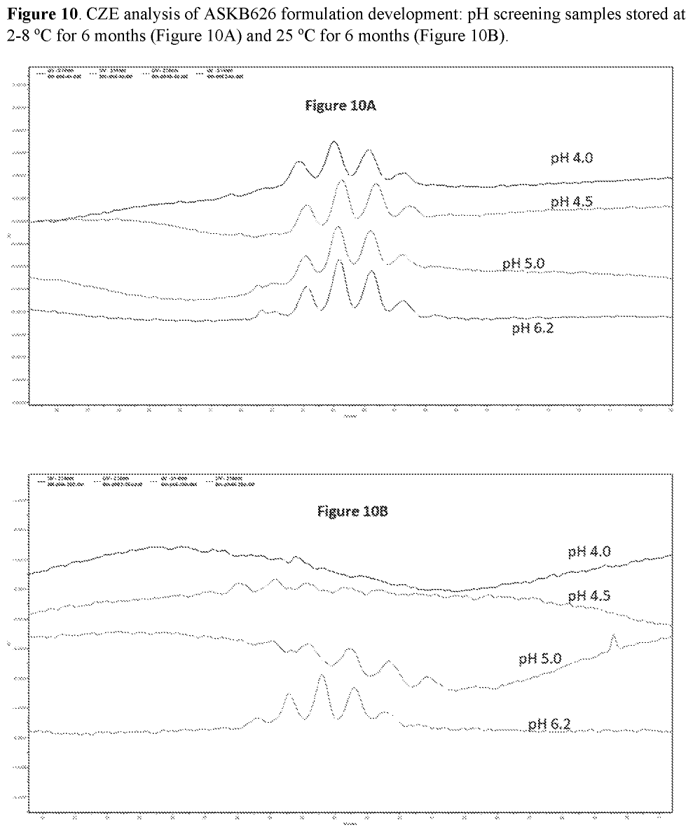

FIG. 10. CZE analysis of ASKB626 formulation development: pH screening samples stored at 2-8° C. for 6 months (FIG. 10A) and 25° C. for 6 months (FIG. 10B).

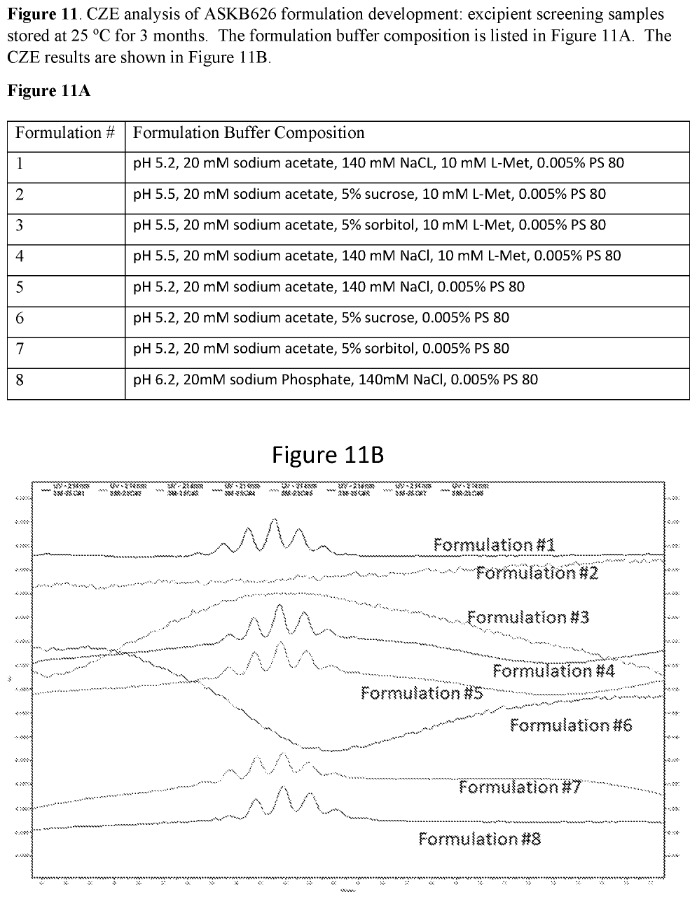

FIG. 11. CZE analysis of ASKB626 formulation development: excipient screening samples stored at 25° C. for 3 months. The formulation buffer composition is listed in

FIG. 11A. The CZE results are shown in FIG. 11B.

### DETAILED DESCRIPTION OF THE INVENTION

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “effective amount” or “therapeutically effective amount” refers to an amount of a compound or composition sufficient to treat a specified disorder, condition, or disease, such as ameliorate, palliate, lessen, and/or delay one or more of its symptoms. In reference to a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

The term “isolated proteins” refers to a protein substantially free of cellular material or culture medium when produced by recombinant DNA techniques, or chemical precursors or other chemicals when chemically synthesized.

The term “sequence homology” or “sequence identity” as used herein refers to the percentage of sequence identity between two polypeptide sequences. In order to determine the percentage of identity between two polypeptide sequences, the amino acid sequences of such two sequences are aligned, preferably using the Clustal W algorithm (Thompson, J D, Higgins D G, Gibson T J, 1994, Nucleic Acids Res. 22 (22): 4673-4680), together with BLOSUM 62 scoring matrix (Henikoff S., and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990: 215: 403). In one aspect the present modified non-human mammalian EPO or fusion proteins have at least 70%, at least 75%, at least 80%, at least 85%, at least 87%, at least 90%, at least 91%, at least 92%, at least 93%, at least 94% at least 95%, at least 96%, at least 97%, at least 98%, at least 99% or 100% sequence identity with another sequence, either on a local or a full-length basis.

If on a local basis, the locality is determined by a region of the non-modified or native sequence, or a specifically identified motif of non-modified or native sequence. In one aspect the locality is at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, or at least 75 nucleic acids or amino acids of the non-modified or native sequence.

The term “variant” as used herein includes modifications or chemical equivalents of the amino acid and nucleotide sequences disclosed herein that perform substantially the same function as the proteins or nucleic acid molecules disclosed herein in substantially the same way. For example, variants of proteins disclosed herein include, without limitation, conservative amino acid substitutions. Variants of proteins disclosed herein also include additions and deletions to the proteins disclosed herein. In addition, variant peptides and variant nucleotide sequences include analogs and chemical derivatives thereof.

The present specification discloses stable sterile pharmaceutical preparations for a recombinant feline erythropoietin (EPO) analog, wherein said recombinant feline EPO analog molecule has an amino acid sequence at least 95% identical as SEQ ID NO: 1 or its modified versions (SEQ ID NO: 2-6). The present specification also discloses pharmaceutical compositions of a recombinant feline EPO analog with enhanced half-life in vivo and reduced risk of immunogenicity, wherein at least 95% of said EPO analog molecules comprise five (5) N-glycans per molecule, at least 85% said EPO analog molecules comprise at least one (1) O-glycans per molecule, and at least 10% of said EPO analog molecules comprise at least two (2) O-glycans per molecule.

In some aspect, the present invention provides stable aqueous formulations for modified feline erythropoietin analog. The recombinant feline EPO analog can have about 95, 96, 97, 98, 99, or 100% identity to SEQ ID NO:1, 2, 3, 4, 5, or 6. In some embodiment, at least 95% of said EPO analog molecules comprise five (5) N-glycans per molecule, at least 85% said EPO analog molecules comprise at least one (1) O-glycans per molecule, and at least 10% of said EPO analog molecules comprise at least two (2) O-glycans per molecule. In some embodiment, at least 95% of the recombinant feline EPO analog proteins contain 19 or more sialic acids per molecule. In some embodiment, at least 90% of the recombinant feline EPO analog proteins contain 20-24 sialic acids per molecule. In some embodiment, the level of aggregation of said recombinant feline EPO analog is less than 2% in the above said stable aqueous formulation.

In an embodiment, the stable formulation comprises polysorbate, NaCl, and phosphate buffer or acetate buffer. The stable formulation may optionally further comprise a stabilizing additive methionine.

In an aspect, the concentration of a therapeutic protein disclosed herein in formulation typically may be between about 0.001 mg/mL to about 2.5 mg/mL. In another embodiment, the concentration is from about 0.01 mg/mL to about 0.10 mg/mL. In another embodiment, the concentration is from about 0.01 mg/mL to about 0.05 mg/mL. In another embodiment, the concentration is approximately 0.025 mg/mL.

In an embodiment, a therapeutically effective amount of a therapeutic protein disclosed herein be from, e.g., about 0.5 microg/KG to about 10 microg/KG, about 1 microg/KG to about 5 microg/KG, about 1 microg/KG to about 2 microg/KG, about 1 microg/KG, about 1.5 microg/KG, or about 2.0 microg/KG.

In one embodiment, the single dose formulation has a protein concentration of about 10 microg/mL to about 60 microg/mL, about 10 microg/mL to about 40microg/mL or about 50 microg/mL to about 125 microg/mL. In one embodiment, the single dose formulation has a concentration of about 10 microg/mL, 25 microg/mL or 50 microg/mL.

In an embodiment, the stable aqueous formulation comprises polysorbate, NaCl and phosphate buffer. In one embodiment, the polysorbate is in a concentration of about 0.02 mg/mL to about 0.2 mg/mL, the NaCl is in a concentration of about 5 mg/mL to about 10 mg/mL and the phosphate buffer is in a concentration from about 5 mM to about 20 mM at pH 5.8-6.5.

In an embodiment, the stable aqueous formulation comprises polysorbate, NaCl and phosphate buffer. In one embodiment, the polysorbate is in a concentration of about 0.02 mg/mL to about 0.2 mg/mL, the NaCl is in a concentration of about 5 mg/mL to about 10 mg/mL and the acetate buffer is in a concentration from about 5 mM to about 20 mM at pH 5.0-6.0.

In one embodiment, the polysorbate is in a concentration of about 0.02 mg/mL to about 0.03 mg/mL, about 0.02 mg/mL to about 0.04 mg/mL, about 0.02 mg/mL to about 0.05 mg/mL, about 0.02 mg/mL to about 0.06 mg/mL, about 0.02 mg/mL to about 0.07 mg/mL, about 0.02 mg/mL to about 0.08 mg/mL, about 0.02 mg/mL to about 0.09 mg/mL, about 0.02 mg/mL to about 0.1 mg/ml, about 0.02 to about 0.11 mg/ml, about 0.02 to about 0.12 mg/ml, about 0.02 to about 0.13 mg/ml, about 0.02 to about 0.14 mg/ml, about 0.02 to about 0.15 mg/ml, about 0.02 to about 0.16 mg/ml, about 0.02 to about 0.17 mg/ml, about 0.02 to about 0.18 mg/ml, or about 0.02 to about 0.19 mg/ml. In one embodiment, the polysorbate is at a concentration of about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, or 0.2 mg/mL.

In one embodiment, the NaCl is in a concentration of about 5 mg/mL to about 10 mg/mL, about 5 mg/mL to about 5.5 mg/mL, about 5 mg/mL to about 6 mg/mL, about 5 mg/mL to about 6.5 mg/mL, about 5 mg/mL to about 7 mg/mL, about 5 mg/mL to about 7.5 mg/mL, about 5 mg/mL to about 8 mg/mL, about 5 mg/mL to about 8.5 mg/mL, about 5 mg/mL to about 9 mg/mL or about 5 mg/mL to about 9.5 mg/mL. In one embodiment, the NaCl is in a concentration of about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL or about 10 mg/mL.

In one embodiment, the phosphate buffer is in a concentration of about 5 mM to about 25 mM, about 5 mM to about 5.5 mM, about 5 mM to about 6 mM, about 5 mM to about 6.5 mM, about 5 mM to about 7 mM, about 5 mM to about 7.5 mM, about 5 mM to about 8 mM, about 5 mM to about 8.5 mM, about 5 mM to about 9 mM about 5 mM to about 9.5 mM, about 5 mM to about 10 mM, about 5 mM to about 20 mM, about 5 mM to about 18 mM, or about 5 mM to about 19.5 mM. In one embodiment, the phosphate buffer is in a concentration of about 5 mM, about 6 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13 mM, about 13. 5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 21 mM, about 22 mM, about 23 mM, about 24 mM, or about 25 mM.

In one embodiment, the acetate buffer is in a concentration of about 5 mM to about 25 mM, about 5 mM to about 5.5 mM, about 5 mM to about 6 mM, about 5 mM to about 6.5 mM, about 5 mM to about 7 mM, about 5 mM to about 7.5 mM, about 5 mM to about 8 mM, about 5 mM to about 8.5 mM, about 5 mM to about 9 mM about 5 mM to about 9.5 mM, about 5 mM to about 10 mM, about 5 mM to about 20 mM, about 5 mM to about 18 mM, or about 5 mM to about 19.5 mM. In one embodiment, the acetate buffer is in a concentration of about 5 mM, about 6 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13 mM, about 13. 5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 21 mM, about 22 mM, about 23 mM, about 24 mM, or about 25 mM.

In one embodiment the pH of the phosphate buffer is from about 5.8 to about 7.0. In one embodiment, the pH is about 5.8, about 5.9, about 6.0, about 6.1, about 6.2, about 6.3, about 6.4, about 6.5, about 6.6, about 6.7, about 6.8, about 6.9, or about 7.

In one embodiment, the aqueous formulation may further comprise methionine. The methionine is at a concentration of about 2 mM to about 20 mM. In one embodiment, the concentration is about 5 mM, about 6 mM, about 7 mM, about 8 mM, about 9 mM, about 10 mM, about 11 mM, about 12 mM, about 13 mM, about 14 mM, about 15 mM, or about 20 mM.

In one embodiment, the aqueous formulation may further comprise feline albumin (for sequence, see, e.g., NP_001009961). The albumin is at a concentration of about 2 mg/mL to about 3 mg/mL. In one embodiment, the concentration is about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3 mg/mL.

In an embodiment, the present modified non-human mammalian erythropoietin or fusion protein may have amino acid additions, deletions, or substitutions. In another embodiment, a modified amino acid sequence is a sequence that is different from the native amino acid sequence due to a deletion, an insertion, a non-conservative or conservative substitution or combinations thereof of one or more amino acid residues. In one embodiment, the modification is a point mutation. In one aspect, the modified non-human mammalian erythropoietin does not have a naturally occurring sequence. Similarly, in one aspect, the P of the fusion protein is a non-naturally occurring amino acid sequence.

In an embodiment, the amino acid substitutions may be conservative or non-conservative. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The most commonly occurring exchanges are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions. Amino acid exchanges in proteins and peptides, which do not generally alter the activity of the proteins or peptides, are known in the art (H. Neurath, R. L. Hill, The Proteins, Academic Press, New York, 1979).

The present modified non-human mammalian EPO or fusion proteins may made by conventional means, such as recombination. The term “recombinant” as used herein refers to a polypeptide produced through a biological host, selected from a mammalian expression system, an insect cell expression system, a yeast expression system, and a bacterial expression system.

N-terminal or C-terminal fusion proteins comprising the proteins disclosed herein conjugated with other molecules, such as proteins may be prepared by fusing, through recombinant techniques. The resultant fusion proteins contain a protein disclosed herein fused to the selected protein or marker protein as described herein. The recombinant protein disclosed herein may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate.

The present disclosures also relate to the treatment of a subject with the modified non-human mammalian EPO or fusion protein disclosed herein. The term “subject” is those suspected of having anemia, which includes but is not limited to mammals including a cat, a dog, a mouse, a rat, a hamster, a rabbit, a guinea pig, a ruminant, a ferret, a non-human primate, a pig, or other livestock having NRA or having the possibility of NRA. In one embodiment, the subject is a cat, a dog, a mouse, a rat, a hamster, a rabbit, a guinea pig, a ruminant, a ferret, a non-human primate, or a pig. However, any non-human subject to be treated with the fusion proteins or the pharmaceutical composition disclosed herein is included without limitation. In an aspect of this embodiment, the subject is not a human being. In another aspect of this embodiment, the treatment of a subject with the modified non-human mammalian EPO or fusion protein disclosed herein is for a veterinary treatment. The pharmaceutical composition including the novel proteins disclosed herein is administered to a subject suspected of anemia, thereby treating the subject effectively.

Since EPO has been shown to have a mitogenic and chemotactic effect on vascular endothelial cells as well as an effect on central cholinergic neurons (see, e.g., Amagnostou et al. (1990) Proc. Natl. Acad. Sci. USA 87:5978-5982 and Konishi et al. (1993) Brain Res. 609:29-35), the compounds disclosed herein may also find use for the treatment of a variety of vascular disorders in subjects, such as promoting wound healing, growth of collateral coronary blood vessels (such as those that may occur after myocardial infarction), trauma, and post-vascular graft treatment, and a variety of neurological disorders, generally characterized by low absolute levels of acetyl choline or low relative levels of acetyl choline as compared to other neuroactive substances e.g., neurotransmitters.

In still another aspect, the present specification provides a pharmaceutical composition (aka, “therapeutic”) for the prevention or treatment of NRA comprising the fusion proteins and modified feline EPO proteins.

The term “formulation” as used herein refers to the recombinant feline EPO analog disclosed herein and excipients combined together which can be administered and has the ability to bind to the corresponding receptors and initiate a signal transduction pathway resulting in the desired activity. The formulation can optionally comprise other agents so long as the present modified non-human mammalian EPO or fusion protein retains the ability to bind the corresponding receptors and ligands.

In an embodiment, a first therapeutic is administered to a subject and at a later date, a second therapeutic is administered to the same subject. In an embodiment, a first therapeutic is administered to a subject at the same time as a second therapeutic is administered to the subject.

In aspects of this embodiment, a sustained release therapeutic delivery platform releases a therapeutic disclosed herein with substantially zero order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release therapeutic delivery platform releases a therapeutic disclosed herein with substantially zero order release kinetics over a period of, e.g., at least 7 days after administration, at least 15 days after administration, at least 30 days after administration, at least 45 days after administration, at least 60 days after administration, at least 75 days after administration, or at least 90 days after administration.

As used herein, the term “prevention” means all of the actions by which the occurrence of the disease is suppressed, restrained or retarded. In the present specification, “prevention” means that the occurrence of anemia is suppressed, restrained or retarded by administration of the conjugates disclosed herein.

As used herein, the term “treatment” means all of the actions by which one or more symptoms of the disease have been alleviated, improved or ameliorated. In the present specification, “treatment” means that the symptoms of NRA are alleviated, improved or ameliorated by administration of the novel proteins disclosed herein.

As used herein, the term “administration” means introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The composition disclosed herein may be formulated into a variety of dosage forms in combination with pharmaceutically acceptable carriers, binders, lubricants, disintegrants, excipients, diluents, solubilizers, dispersing agents, stabilizers, suspending agents, colorants, flavorants, buffering agents, preserving agents, anti-oxidants, analgesics, solubilizers, isotonic agents, and base materials.

For injectable preparations, the pharmaceutical composition may be formulated into an ampule as a single dosage form or a multi-dose container. The pharmaceutical composition may also be formulated into solutions, suspensions, tablets, pills, capsules and long-acting preparations. For example, for oral administration, the pharmaceutical composition may be formulated into tablets, troches, capsules, elixirs, suspensions, syrups or wafers.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The pharmaceutical composition disclosed herein may further include a pharmaceutically acceptable excipient or diluent. As used herein, the term “pharmaceutically acceptable” means that the composition is sufficient to achieve the therapeutic effects without deleterious side effects, and may be readily determined depending on the type of the diseases, the subject's species, age, body weight, health conditions, gender, and drug sensitivity, administration route, administration mode, administration frequency, duration of treatment, drugs used in combination or coincident with the composition disclosed herein, and other factors known in medicine.

For injectable preparations, the pharmaceutical composition may include a buffering agent, a preserving agent, an analgesic, a solubilizer, an isotonic agent, and a stabilizer. For preparations for topical administration, the carrier may include a base, an excipient, a lubricant, and a preserving agent. For oral administration, the pharmaceutical composition may include, but is not limited to, a carrier, a binder, a lubricant, a disintegrant, an excipient, a diluent, a solubilizer, a dispersing agent, a stabilizer, a suspending agent, a colorant, and a flavorant.

Examples of the carrier for the pharmaceutical compositions include physiological saline, organic solvents, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the excipient or diluent for pharmaceutical formulations may further include fillers, anti-coagulating agents, solubilizer, anti-oxidants, lubricants, humectants, flavorants, and antiseptics. In one embodiment, the pharmaceutical composition may be obtained by blending with a variety of pharmaceutically acceptable carriers. In order to increase the stability or absorptivity, carbohydrates such as glucose, sucrose or dextrans, antioxidants such as ascorbic acid or glutathione, chelating agents, low molecular weight proteins or other stabilizers may be used.

Further, the pharmaceutical composition may be formulated into a single dosage form suitable for the subject's body. For example, the pharmaceutical composition is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The administration dose and frequency of the pharmaceutical composition disclosed herein are determined by the type of active ingredient, together with various factors such as the disease to be treated, administration route, subject's species, age, gender, and body weight, and disease severity.

Methods disclosed herein include administration of the recombinant feline EPO analog disclosed herein prior to, substantially contemporaneously with or after the subject has been diagnosed with NRA, and administration prior to, substantially contemporaneously with or after a pathology or a development of one or more adverse symptoms of NRA or pathologies caused by NRA. Methods, compositions and uses disclosed herein also include administration of the modified non-human mammalian EPO or fusion proteins to a subject prior to, substantially contemporaneously with or following the identification of an adverse symptom, disorder, illness or disease caused by or associated with NRA, or pathology resulting from NRA. A subject with NRA may have NRA or present symptoms of NRA over a period of 1-5, 5-10, 10-20, 20-30, 30-50, 50-100 hours, days, weeks, months, or years.

The compositions disclosed herein can be combined with any compound, agent, drug, treatment or other therapeutic regimen or protocol having a desired therapeutic, beneficial, additive, synergistic or complementary activity or effect. Exemplary combination compositions and treatments include second actives such as, e.g., drugs or agents related to the treatment of bone marrow disease, tick disease, abscesses, cancer, kidney failure, toxic chemical exposure, radiation exposure, lead poisoning, and inherited NRA. Such drugs, agents, treatments and therapies can be administered or performed prior to, substantially contemporaneously with or following any other method disclosed herein, for example, a therapeutic method of treating a subject for an NRA thereto, or a method of prophylactic treatment of a subject for NRA. Moreover, the pharmaceutical composition may be administered alone or in combination or coincident with other pharmaceutical formulations showing prophylactic or therapeutic effects on anemia, especially NRA.

The compositions disclosed herein can be administered as a combination composition, or administered separately, such as concurrently or in series or sequentially (prior to or following) administering a second active, to an individual. The present specification therefore provides combinations in which a method or use is used in a combination with any compound, agent, drug, therapeutic regimen, treatment protocol, process, remedy or composition, such as drugs or agents related to the treatment of bone marrow disease, tick disease, abscesses, cancer, kidney failure, toxic chemical exposure, radiation exposure, lead poisoning, and inherited NRA. The compound, agent, drug, therapeutic regimen, treatment protocol, process, remedy or composition can be administered or performed prior to, substantially contemporaneously with or following administration of one or more of the present modified non-human mammalian EPO or fusion proteins or subsequences, portions or modifications thereof, or a nucleic acid encoding all or a portion of the present modified non-human mammalian EPO or fusion proteins, subsequence, portion or modification thereof, to a subject. Specific non-limiting examples of combination embodiments therefore include the foregoing or other compound, agent, drug, therapeutic regimen, treatment protocol, process, remedy or composition.

The total effective dose of the composition disclosed herein may be administered to a subject in a single dose, or may be administered for a period of time in multiple doses according to a fractionated treatment protocol. For instance, the dose may be determined as a total dose over the lifetime of the subject, a total dose over the expected treatment period, a total monthly dose, a total weekly dose, or a total daily dose.

In an embodiment, the period of administration of a modified non-human mammalian EPO or fusion proteins disclosed herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Dosing can be single dosage or cumulative (serial dosing), and can be readily determined by one skilled in the art. For instance, treatment of anemia may comprise a one-time administration of a sufficient dose of a composition disclosed herein. As a non-limiting example, a sufficient dose of a composition disclosed herein can be administered once to a subject, e.g., as a single injection or deposition or a single oral administration. Alternatively, treatment may comprise a one-time administration of a sufficient dose of a composition disclosed herein carried out over a range of time periods, such as, e.g., daily, once every few days, weekly, monthly or yearly. As a non-limiting example, an antigen or a composition disclosed herein can be administered once or twice weekly to a subject. The timing of administration can vary from subject to subject, depending upon such factors as the severity of the subject's symptoms. For example, a sufficient dose of an antigen or a composition disclosed herein can be administered to a subject once a month for an indefinite period of time, or until the subject no longer requires therapy. A person of ordinary skill in the art will recognize that the condition of the subject can be monitored throughout the course of treatment and that the sufficient amount of a composition disclosed herein that is administered can be adjusted accordingly.

In one embodiment the total daily dose of the recombinant feline EPO analog disclosed herein may be approximately 0.01 μg to 0.25 mg per 1 kg of body weight of a patient (aka μg/kg or mg/kg). In one embodiment, the total daily dose is at least 0.01 μg/kg, at least 0.05 μg/kg, at least 0.1 μg/kg, at least 0.5 μg/kg, about 1 μg/kg, about 1.5 μg/kg, about 2 μg/kg, about 2.5 μg/kg, about 5 μg/kg, about 0.01 mg/kg, about 0.015 mg/kg, about 0.025 mg/kg, or about 0.05 mg/kg. In one aspect, the maximum daily dose is at most at most 0.05 mg/kg, at most 0.025 mg/kg, at most 0.015 mg/kg, at most 10 μg/kg, at most 5 μg/kg, at most 1 μg/kg, at most 0.05 μg/kg, or at most 0.1 μg/kg. In yet another aspect the daily dose of the modified non-human mammalian EPO or fusion protein disclosed herein may be approximately 0.05 μg/kg to 0.05 mg/kg, 0.1 μg/kg to 0.01 mg/kg, 1 μg/kg to 0.005 mg/kg, or 50 μg/kg to 0.1mg/kg. In still other aspects of this embodiment, a sufficient amount of a modified non-human mammalian EPO or fusion protein disclosed herein is the dosage sufficient to reduce a symptom associated with anemia for, e.g., at least one week, at least one month, at least two months, at least three months, at least four months, at least five months, at least six months, at least seven months, at least eight months, at least nine months, at least ten months, at least eleven months, or at least twelve months.

In an embodiment, the effective dose of the recombinant EPO analog is determined considering various factors including subject's species, age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. In a further embodiment, the pharmaceutical composition disclosed herein is not particularly limited to the formulation, and administration route and mode, as long as it shows a beneficial effects.

In an embodiment, the pharmaceutical composition disclosed herein is expected to have longer in vivo duration of efficacy and titer, thereby remarkably reducing the number and frequency of administration thereof when compared to administration of native EPO (i.e., EPO having the full-length amino acid sequence of the naturally occurring EPO amino acid sequence). In one embodiment, a composition disclosed herein and its derivatives or variants have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, an individual is provided a treatment protocol wherein a pharmaceutical composition is to be administered to a subject on a periodic schedule, wherein the individual is informed by electronic notification to administer the therapeutic on a period schedule. In an aspect of this embodiment, the electronic notification is by email, text, instant messaging or by another electronic notification method. In an embodiment, an individual is informed to administer the presently disclosed therapeutic on a period schedule through receipt of a telephone call, postal mail, overnight express (including, without limitation, FedEx and UPS) or other method of notification.

In order that this invention may be better understood, the following examples are set forth. These examples are for purposes of illustration only and are not to be construed as limiting the scope of the invention in any manner.

In other aspects of this embodiment, a nonhuman, mammalian EPO disclosed herein reduces the severity of NRA by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a nonhuman, mammalian EPO disclosed herein reduces the severity of NRA from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A nonhuman, mammalian EPO disclosed herein may comprise a therapeutic compound in an amount sufficient to allow customary administration to a nonhuman mammal, including a cat and with other excipients may constitute a pharmaceutical composition.

A nonhuman, mammalian EPO disclosed herein may comprise a solvent, emulsion or other diluent in an amount sufficient to dissolve a nonhuman, mammalian EPO disclosed herein. In other aspects of this embodiment, a nonhuman, mammalian EPO disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a nonhuman, mammalian EPO disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

Aspects of the present specification disclose, in part, treating a nonhuman mammalian individual suffering from a disease, including NRA. As used herein, the term “treating,” refers to reducing or eliminating in a nonhuman, mammalian a clinical symptom of NRA; or delaying or preventing in a nonhuman, mammalian the onset of a clinical symptom of NRA. For example, the term “treating” can mean reducing a symptom of a condition characterized by NRA, including, but not limited to, reduction of the severity of the disease, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with NRA are well known and can be determined by a person of ordinary skill in the art by taking into account factors, including, without limitation, the location of the disease, the cause of the disease, the severity of the disease, and/or the tissue or organ affected by the disease. Those of skill in the art will know the appropriate symptoms or indicators associated with a specific type of disease, and will know how to determine if an individual is a candidate for treatment as disclosed herein.

In aspects of this embodiment, a therapeutically effective amount of a nonhuman, mammalian EPO disclosed herein reduces a symptom associated with a disease, including NRA by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a nonhuman, mammalian EPO amount of a therapeutic compound disclosed herein reduces a symptom associated with a disease, for instance, NRA by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a nonhuman, mammalian EPO disclosed herein reduces a symptom associated with a disease, including NRA by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

Dosing can be single dosage or cumulative (serial dosing), and can be readily determined by one skilled in the art. For instance, treatment of a disease, including NRA may comprise a one-time administration of an effective dose of a nonhuman, mammalian EPO disclosed herein. Alternatively, treatment of a disease, including NRA may comprise multiple administrations of an effective dose of a nonhuman, mammalian EPO carried out over a range of time periods, such as, e.g., once daily, twice daily, trice daily, once every few days, or once weekly. The timing of administration can vary from nonhuman, mammalian to nonhuman, mammalian, depending upon such factors as the severity of a nonhuman, mammalian's symptoms. For example, an effective dose of a nonhuman, mammalian EPO disclosed herein can be administered to an individual once daily for an indefinite period of time, or until the nonhuman, mammalian no longer requires therapy. A person of ordinary skill in the art will recognize that the condition of the nonhuman, mammalian can be monitored throughout the course of treatment and that the effective amount of a nonhuman, mammalian EPO disclosed herein that is administered can be adjusted accordingly.

In one embodiment, a nonhuman, mammalian EPO disclosed herein is capable of reducing the incidence of NRA in a nonhuman, mammalian by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a nonhuman, mammalian EPO is capable of reducing the incidence of NRA in a nonhuman, mammalian suffering from NRA by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a nonhuman, mammalian EPO and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a nonhuman, mammalian EPO is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a nonhuman, mammalian EPO disclosed herein reduces or maintains a disease, including NRA in a nonhuman, mammalian by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a nonhuman, mammalian EPO disclosed herein reduces or maintains a disease, including NRA in a nonhuman, mammalian by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a nonhuman, mammalian EPO disclosed herein reduces or maintains a disease, including NRA in a nonhuman, mammalian by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A nonhuman, mammalian EPO is administered to an individual. An individual is typically a nonhuman, mammalian, including, but not limited to, dogs, cats, birds, cattle, horses, sheep, goats, reptiles and other animals, whether domesticated or not. Typically, any nonhuman, mammalian who is a candidate for treatment is a candidate with some form of disease, including NRA. Pre-operative evaluation typically includes routine history and physical examination in addition to thorough informed consent disclosing all relevant risks and benefits of the procedure.

### EXAMPLES

### Example 1

### Production of Recombinant Feline EPO Analog (fEPO Analog)

Cloning: Vector construction was carried out by DNA 2.0. The DNA for the feline EPO analog (fEPO analog with the amino acid sequence as shown in SEQ ID NO: 1 and DNA sequence as shown in SEQ ID NO: 7) coding sequence with signal peptide and endonuclease restrict sequence (5′-Hind III, 3′-Pac I) was synthesized. The synthesized DNA were cloned into an expression vector PCGS3 (from Sigma-Aldrich®) between the Hind III and Pac I sites. The complete plasmid with the modified feline EPO DNA gene (named as ASKBH01) was confirmed by fully DNA sequencing. The plasmid was transformed into DH10B E. coli. DNA was prepared and purified by endo-free plasmid kit (from Qiagen®).

Cell line development: The fEPO analog plasmid DNA were prepared and purified by using endo-free plasmid Maxi kit from Qiagen®. The host cell line CHOZN® ZFN-Modified GS−/− CHO cells from Sigma-Aldrich® were cultured in complete EX-CELL® CD CHO Fusion Growth Medium supplemented glutamine. The transfection was performed by electroporation (Bio-Rad Gene Pulser®). The transfected cells were screened and selected by using EX-CELL® CD CHO Fusion Growth Medium without glutamine. 32 stable minipools were established, the leading mini-pool was selected based on expression level in batch and fed-batch culture. Single cloning was performed by limited dilution and using clone media, two leading single clones out of 132 positive clones (in total 960 wells) were selected based on productivity and cell growth in batch and fed-batch culture.

Fed-batch cell culture: The lead clones were expanded and seeded at 0.5×106 cells/ml, total 300 ml in 2 L shake flasks, and the cells were cultured at 37° C., 5% CO2, 70% HMR conditions and shaking at 120 rpm. The cultures were fed by using 5% Acti CHO® Feed A+0.5% Feed B (from GE Health) on day 3,6,7,8 and 9. The cell viability and viable cell density were monitored every other day, the cultures were harvested on day 11. The cell growth profile, viability and titer are shown in FIG. 9.

Harvesting: Approximately 600 ml of the cultured cell medium was clarified through centrifugation at 2000 RPM for 10 min followed by filtration. The clarified supernatant was concentrated to approximately 100 ml and buffer exchanged into 10 mM Tris, pH 7.1. A Pellicone II UF membrane with 30 KD molecular weight cut-off was used for this UFDF step. The UFDF pool was aliquoted and frozen at −80° C. until further purification.

Initial purification: The UFDF pool containing the modified feline EPO was thawed and loaded to a column packed with Q sepahrose FF resin at neutral pH. The column has a diameter of 2 cm and a bed height of approximately 1.5 cm. The column was washed with 10 mM Tris, pH 7.1, followed by a 2^{nd} wash with 2 mM acetic acid, 1 mM glycine, 6 M urea, 20 micoM CuSO4, pH 4.8 to remove host cell impurities and feline EPO isoforms with approximately 7 sialic acids or less. The column was further washed with 40 mM acetic acid, 1 mM glycine, 6 M urea, 20 micoM CuSO4, pH 4.0. This step removed additional cell impurities and feline EPO isoforms containing approximately 8 to approximately 12 sialic acids. The column was then eluted with 10 mM Tris, 140 mM NaCl, 20 micoM CuSO4, pH 7.1 to elute the modified feline EPO isoforms containing more than approximately 12 sialic acids.

Further purification: The elution pool containing 12 or more sialic acids is further purified using reverse phase chromatography, a 2^{nd} anion exchange chromatography, size exclusion chromatography, multimodal weak cation exchanger, multimodal weak anion exchanger, and/or hydroxyapatite chromatography to achieve high purity, wherein the level of aggregates is lower than 0.5%, preferably <0.2%, and further preferably <0.1%.

### Example 2

### SEC-HPLC Analysis

SEC-HPLC was carried out using an Agilent 1100 Series of HPLC system with a TSKgel G3000SWXL column (7.8 mmlDX 30 cm, 5 μm particle size, part #0008541) ordered from Tosoh Bioscience. A sample of up to 100 μl was loaded. The column was run with a buffer containing 1.5 mM potassium phosphate monobasic, 8.1 mM sodium phosphate dibasic, 400 mM NaCl, pH 7.4. The flow rate was 0.5 ml/min. The column was run at room temperature. The protein elution was monitored both at 220 nm and 280 nm. A representative SEC-HPLC chromatograph is shown in FIG. 8. The result showed that the purified ASKB626 sample had no or very little aggregates.

### Example 3

### IEF Analysis

IEF is used to assess the charge isoforms of ASK-B626 molecules associated with the negatively charged glycans. HPE™ BlueHorizon^{™} Flat-bed IEF System is used with the EPO Doping IEF Kit 30S from Serva Electrophoresis. The IEF was run following the procedure below:
a) Set chiller temperature to 10° C.
b) Apply 5 mL of cooling fluid on the HPE™ BlueHorizon™ flat bed. Carefully lay rehydrated 30S gel on the bed. Remove air bubbles between the bed surface and gel bottom by slowly roll away the air bubbles. Sample wells are on the bottom of the gel.
c) Use filter paper to remove the extra hydration buffer on the surface of the gel and sample wells
d) Cut two electrode wicks just longer than the gel length. Wet one wick in cathode fluid and one wick in anode fluid. Carefully place soaked cathode wick on the bottom of the gel (between the lower bottom and sample well). Carefully place the soaked anode wick on the top of the gel. Then close the IEF lid. Make sure the red strip touches the anode wick, and the black strip touches the cathode wick.
e) Pre-focus gel at 250V/30 mA/30 W for 30 min
f) Apply 10 μL of the standard and 10 μL of each samples on the gel well. The samples and standard should have a protein concentration of approximately 0.5 mg/ml.
g) Focus gel at 2000V/50 mA/30 W until 4000V·h has been reached (2-2.5 hours)

The IEF gel was fixed and stained following the procedure below following the procedure below
a) Carefully remove the electrode wicks after electrophoresis with tweezers. Transfer gel immediately into a tray containing 20% trichloroacetic acid. Shake for 20 min.
b) Rinse gel with DI water for 1 min. Repeat one more time.
c) Stain gel in SERVA Blue W for 20 min by mild shaking.
d) Rinse with DI water for 2-3 min to destain the gel until clear blue band shows up. If the background is too dark, dilute fixation fluid from 20% (w/v) trichloroacetic acid to 0.4% (w/v), and then destain the gel for 30 sec or until background blue has been removed.

Electropherogram of Aranesp® and ASKB626 DS sample is shown in FIG. 3. Multiple charged isoforms are observed in ASK-B626 and Aranesp®, which are characteristic of the molecules. It was discovered that two major bands of the ASKB626 sample were running at or below the last major band of Aranesp®, wherein the last band is assumed to contain 22 sialic acids per molecule.

### Example 4

### CZE Analysis

CZE analysis was carried out essentially as described by Zhang et al. (Anal. Chem., 2015, 87 (1), pp 470-476). Briefly, the method was run with Sciex PA 800 Plus instrument with 32 Karat software. The capillary was treated with 0.1 N NaOH, rinsed with distilled water, rinsed again with 0.1 N NaOH, and then equilibrated with the CZE separation buffer, which contains 7 M urea, 2.5 mM Putrescine, 10 mM Tricine, 10 mM NaCl, 6 mM Sodium Acetate, pH 4.5. The voltage during equilibration was set at 20 kV and the equilibration was run for 720 min. The sample was loaded and the separation was run at voltage of 15.7 kV for 100 min.

A representative chromatograph of the CZE analysis of ASKB626 and Aranesp® is shown in FIG. 4. It was discovered that three of the four most abundant peaks of ASKB626 migrate slower than the slowest peak of the four most abundant peaks of Aranesp®. The results indicated that ASKB646 contained species that had more than 22 sialic acids per molecule , the highest number of sialic acids possible for Aranesp®.

### Example 5

### Peptide Mapping-Based N-Glycan Occupation Analysis

### Experimental Principle

The site occupancy for N-linked glycans was estimated by deglycosylating the protein with PNGase F, digesting with trypsin, and analyzing by nUHPLC-MS/MS. Since PNGase F converts glycosylated asparagine to aspartic acid, it is possible to estimate the site occupancy by comparing the level of asparagine to aspartic acid at sites containing the consensus N-linked glycosylation motif (NX(S/T), where N is asparagine, X is any amino acid except proline, and (S/T) is either serine or threonine). The background level of deamidation can be estimated by comparing the level of asparagine to aspartic acid at a site that does not contain the consensus N-linked glycosylation motif.

### Deglycosylation

The sample was deglycosylated using PNGase F (New England Biolabs) under denaturing conditions following the manufacturer's protocol, except that the reaction was allowed to continue overnight instead of for 1 hour. The deglycosylated protein was then cleaned up using a detergent removal column (Thermo Pierce) to remove SDS and NP-40 used in the deglycosylation protocol and to exchange it into 50 mM ammonium bicarbonate.

### Digestion

The deglycosylated protein was reduced with DTT and alkylated with iodoacetamide. The deglycosylated and alkylated protein was divided into three fractions. One fraction was acidified to pH ˜2.5 using 1M HCI, and pepsin was added at an approximately 1:20 pepsin:protein ratio. The sample with pepsin added was allowed to digest for 1 hour, after which the pepsin was inactivated by raising the pH by addition of 1M NH₄HCO₃. The second and third fractions were digested using trypsin and chymotrypsin respectively, also at a roughly 1:20 enzyme:protein ratio, but the reactions were allowed to continue overnight.

### Liquid Chromatography/Mass Spectrometry

The samples were analyzed by nUHPLC-MS/MS using an EasyNano1000 UHPLC and Orbitrap Fusion mass spectrometer (both Thermo Scientific). Samples were loaded onto a 2cm×75 μm ID trapping column packed with 3 μm PepMap 100 C₁₈ silica at 5 μl/min, then eluted through a 25cm×75 μm ID analytical column packed with 2 μm PepMap 100 C₁₈ silica at 300 nl/min. Buffers A and B were 0.1% formic acid in water and acetonitrile, respectively. Peptides were eluted using a linear gradient from 3%-8% buffer B over 5 minutes, then from 8% to 38% B over 90 minutes, and 38%-90% B over 1 minute.

Column eluate was sprayed directly into the mass spectrometer using the EasySpray source at a spray potential of 2300V. Data was acquired using a data-dependent program. Full mass range spectra were acquired in the Orbitrap portion of the instrument at 120,000 nominal resolution and a scan range of 400-1600 using quadrupole isolation and the instrument's internal calibrant delivery system for improved mass accuracy. From each full mass range spectrum, a set of targets were chosen for tandem MS analysis, with a maximum time between full mass range scans of 2 seconds. Targets were chosen using monoisotopic precursor selection using parameters appropriate for peptides and filtered for a minimum intensity of 5×10³ counts and charge state between 2 and 7. After acquiring one tandem mass spectrum, targeted ions were placed on an exclusion list for 20 seconds. Target ions were fragmented using CID with 35% relative collision energy and analyzed in the orbitrap analyzer at 30,000 nominal resolution.

### Data Analysis

Mass spectra were assigned to the protein sequence using Sequest HT in Proteome Discoverer (Thermo Scientific) using a database containing the sequence of interest and a set of common laboratory contaminant proteins. The search used a precursor mass tolerance of 5 ppm, a fragment mass tolerance of 0.02 Da, and no enzymatic specificity. It assumed quantitative carbamidomethylation of cysteine and considered possible oxidation of methionine, deamidation of asparagine, acetylation of the protein amino terminus, and glycosylation of serine and threonine. PTMrs was used to localize the site of modification for each identified modification. The software was configured to integrate and report precursor ion area for each precursor ion.

### Results

A database search identified peptides that contained all 7 of the asparagines found in the sequence, including all of the asparagines that are part of the consensus N-linked glycosylation motif. For each identified asparagine, precursor ions for peptides containing that asparagine were classified as modified or unmodified depending on whether the asparagine was determined to be deamidated or not. The integrated precursor ion intensities for each category were summed to create an overall intensity for modified and unmodified forms of that asparagine. The overall intensities were then converted to an estimated site occupancy defined as:

Site occupancy=100%*modified intensity/(modified intensity+unmodified intensity)

Calculations were performed separately for each digestion condition so the different forms can be used as a cross check on each other. Results are summarized in Table 1.

TABLE 1
Summary of site occupancy for identified asparagine residues. The
“Control” value represents a weighted average of the values
for positions 47 and 147, which are not expected to be glycosylated.

| | pH 4.5 | | | pH 5.0 | | |
|---|---|---|---|---|---|---|
| | Chymo- | | | Chymo- | | |
| Position | trypsin | Pepsin | Trypsin | trypsin | Pepsin | Trypsin |
| 24 | 99.9% | 100.0% | 100.0% | 95.2% | 99.2% | 100.0% |
| 30 | 99.8% | 99.1% | 100.0% | 95.0% | 97.5% | 100.0% |
| 38 | 98.9% | 100.0% | 99.9% | 97.7% | 98.2% | 99.8% |
| 47 | 12.8% | 0.0% | 2.4% | 15.6% | 0.0% | 2.3% |
| 83 | 100.0% | 100.0% | 100.0% | 100.0% | 100.0% | 100.0% |
| 88 | 99.9% | 99.8% | 100.0% | 99.7% | 99.7% | 100.0% |
| 147 | 46.4% | 7.3% | 0.0% | 56.7% | 100.0% | 0.0% |
| Control | 13.0% | 0.2% | 2.3% | 16.2% | 0.2% | 2.3% |

All asparagines that are predicted to be N-linked glycosylation sites (24, 30, 38, 83, and 88) were detected in each digest, and they were identified either predominantly or exclusively in the deamidated form. Peptides containing asparagines 47 and 147, which are not predicted glycosylation sites, were also detected, and with a lower level of deamidation. The level of artifact deamidation was notably higher for the chymotrypsin digested sample than for either the pepsin or trypsin sample. It is possible to correct for the level of background deamidation using the formula:

Corrected value=1−(1−uncorrected value)/(1−uncorrected N47+N147)

where “uncorrected N47+N147” represents the weighted average deamidation level for asparagines 47 and 147. Corrected data for the samples is shown in Table 2. The correction factor does not change the qualitative result: the predicted N-linked glycosylation sites are either predominantly or completely occupied.

TABLE 2
Calculated site occupancy for asparagines that
are part of the consensus N-linked glycosylation motif
after correction for background deamidation.

| | Enzyme | | |
|---|---|---|---|
| Position | Chymotrypsin | Pepsin | Trypsin |
| 24 | 99.89% | 100.00% | 100.00% |
| 30 | 99.81% | 99.06% | 100.00% |
| 38 | 98.75% | 100.00% | 99.86% |
| 83 | 100.00% | 99.99% | 100.00% |
| 88 | 99.85% | 99.77% | 100.00% |

### Example 6

### Peptide Mapping-Based O-Glycan Occupation Analysis

### Materials and Methods

A total of 200 μL of the drug substance ASKB626/DS/LL14-36; 0.55 mg/ml, was digested using a combination of PNGaseF and sialidase at 37° C. for overnight. The digested sample was first analyzed by LC-MS using an Agilent 1290 UPLC system and an Advance Bio LC/Q-TOF mass spectrometer. Approximately 10 μg of the sample was injected onto a reversed-phase C4 column and then eluted off the samples and injected into the mass spectrometer. Mobile phase A is composed of 0.1% TFA in water and mobile phase B is composed of 0.1% TFA in acetonitrile. The column was heated at 60° C.

The deglycosylated and desialylated samples were further digested using either Lys-C alone or a combination of Lys-C and trypsin at 1:5 enzyme to protein ratio at 37° C. for overnight. The same UPLC and mass spectrometer were used to analyze the peptides. Approximately 10 μg of each digested samples was injected onto a reversed phase C18 column and eluted using a gradient of increasing mobile phase B. The same mobile phases used for intact molecular weight analysis were used for peptide analysis. The column temperature was set at 60° C.

### Results and Discussion

### Intact Molecule Weight Analysis

The sample after PNGaseF and sialidase digestion was first analyzed by LC-MS to analyze the level of O-linked glycosylation. The calculated molecular weights of the molecule are shown in Table 3. When calculating the theoretical molecular weights, several factors were taken into consideration. First, deglycosylation of the N-linked oligosaccharides by PNGaseF converts the original asparagine to aspartate, which increases the molecular weight by a total five Dalton because of the presence of five N-linked oligosaccharides. Secondly, the C-terminal arginine residue is absent from the molecule due to posttranslational modifications. Third, each site of O-linked oligosaccharides with the common disaccharides, HexNAcHex, increases the molecular weight by 365 Da.

TABLE 3
Amino acid sequences and calculated molecular
weights

| | Calculated molecular |
|---|---|
| Amino acid sequences | weights |
| APPRLICDSR VLERYILEAR | No O-linked oligosac- |
| EAENVTMGCN | charides: 18320 Da |
| ETCSFSENIT VPDTKVNFYT | With one O-linked oligo- |
| WKRMDVGQQA | saccharide: 18685 Da |
| VEVWQGLALL SEAILRGQAL | With two O-linked |
| LANSSQVNET LQLHVDKAVS | oligosaccharides: |
| SLRSLTSLLR ALGAQKEATS | 19050 Da |
| LPEATSAAPL RTFTVDTLCK | With three O-linked |
| LFRIYSNFLR GKLTLYTGEA | oligosaccharides: |
| CRRGD ® | 19415 Da |

The data from analysis of the drug substance sample are shown in FIG. 5. As shown in the total ion chromatogram (TIC), four peaks or shoulders were detected and labeled as P1 to P4. The molecular weight of the major peak in P1 is 19415 Da, which corresponds to the molecule with three HexNAcHex. The major peak in P2 and P3 is 19050 Da, which corresponds to the molecule with two HexNAcHex. The molecular weight of the major peak in P4 is 18685 Da, which corresponds to the molecule with one HexNAcHex.

The relative intensities of the molecules with different numbers of O-linked oligosaccharides, the summed mass spectrum that includes P1-P4 are shown in FIG. 6. The relative percentage of the molecule with different numbers of O-linked oligosaccharides is also compared by calculating the percentage of each form using the peak intensities and shown in Table 4. The species with HexNAc or without O-linked oligosaccharides can be generated due to the loss of sugars caused by in-source fragmentation.

TABLE 4
Relative percentage of the molecule with different
numbers of O-linked oligosaccharides

| | Percentage of Molecules with various |
|---|---|
| Species | levels of O-linked glycosylation |
| No O-linked | 8.5% |
| With HexNAc* | 6.2% |
| 1 O-linked | 38.0% |
| 2 O-linked | 35.0% |
| 3 O-linked | 12.3% |

*Could be due to in-source fragmentation

### Peptide Mapping

To determine the location of the O-linked oligosaccharide and further compare the relative percentage, the samples after PNGaseF and sialidase digestion were digested using Lys-C. The summary of the peptide mapping results is shown in Table 5. All the expected peptides were detected. Peptides with amino acids 1-45, or 53-97 contain three or two sites of N-linked glycosylation as indicated by molecular weight increases of 3 or 2 Da compared to their respective calculated molecular weights due to the conversion of asparagine to aspartate from PNGaseF digestion. The C-terminal peptide was detected with the absence of the last arginine residue. Up to three sites of O-linked oligosaccharides were detected to associate with the peptide with amino acids 117-140, supported by a molecular weight increment of approximately 365 Da for each site of O-glycosylation.

TABLE 5
Calculated and observed Lys-C peptide
molecular weights

| | | Calculated | Observed molecular | |
|---|---|---|---|---|
| | | molecular | weights (MH+) Da | |
| Posi- | Amino acid | weights | Drug | In- |
| tion | sequence | (MH+) Da | substance | process |
| 1-45 | APPRLICDSR | 5070.4 | 5073.4 | 5073.4 Da |
| | VLERYILEAR | | Three | Three |
| | EAENVTMGCN | | sites | sites of |
| | ETCSFSENIT | | of N- | N-linked |
| | VPDTK | | linked | glyco- |
| | | | glyco- | sylation |
| | | | sylation | |
| 46-52 | VNFYTWK | 957.5 | 957.5 | 957.5 |
| 53-97 | RMDVGQQAVE | 4928.6 | 4930.6 Da | 4930.6 Da |
| | VWQGLALLSE | | Two sites | Two sites |
| | AILRGQALLA | | of N- | of N- |
| | NSSQVNETLQ | | linked | linked |
| | LHVDK | | glyco- | glyco- |
| | | | sylation | sylation |
| 98-116 | AVSSLRSLTS | 1971.2 | 1971.2 Da | 1971.2 Da |
| | LLRALGAQK | | | |
| 117-140 | EATSLPEATS | 2522.3 (0) | 2522.3 | 2522.3 |
| | AAPLRTFTV | 2887.4 (1) | 2887.4 | 2887.4 |
| | DTLCK | 3252.5 (2) | 3252.6 | 3252.6 |
| | | 3617.6 (3) | 3617.7 | 3617.7 |
| | | Number of | | |
| | | O-linked | | |
| | | indicated | | |
| | | in paren- | | |
| | | thesis | | |
| 141-152 | LFRIYSNFLR | 1513.9 | 1513.9 Da | 1513.9 Da |
| | GK | | | |
| 153-165 | LTLYTGEACR | 1454.7 | 1454.7 Da | 1454.7 Da |
| | RGDR | | Absence of | Absence of |
| | | | C-terminal | C-terminal |
| | | | R | R |

The extracted ion chromatograms and the corresponding mass spectrum are shown in FIG. 7. Interestingly, there are two peaks corresponding to the peptide with 2 O-linked oligosaccharides, suggesting the specific location of O-glycosylation can influence the peptide retention times. The peptide without O-linked oligosaccharide was detected at an extremely low amount and was not taken into consideration when calculating the relative percentage of each peptide.

The relative percentage of the peptide with different numbers of O-linked oligosaccharides was calculated based on the EIC peak areas and is summarized in Table 5. The Lys-C digested sample was further digested using trypsin and analyzed by LC-MS. Analysis from trypsin digestion localized the observed O-linked oligosaccharides to amino acids 117-131. The relative percentage of the peptide with different numbers of O-linked oligosaccharides is also shown in Table 6. The data from Lys-C and both Lys-C and trypsin digestion are in good agreement.

TABLE 6
The relative percentage of one, two or
three O-linked oligosaccharides

| | Drug substance | |
|---|---|---|
| Species | Lys-C | Trypsin |
| 1 O-linked | 39.6% | 42.3% |
| 2 O-linked a | 36.1% | 35.5% |
| 2 O-linked b | 8.5% | 7.2% |
| 3 O-linked | 15.8% | 14.9% |

There were more than 40%of the recombinant feline EPO analog (ASKB626) molecules in the drug substance that contained two or three O-linked glycans.

### Example 7

### Formulation Development—pH Screening

PH Screening Study is designed to have four pHs, pH 4.0, pH 4.5, pH 5.0 and pH 6.2. The formulations are listed below:
1. pH 4, 10 mM sodium acetate, 140 mM NaCl, 0.007% PS 80
2. pH 4.5, 10 mM sodium acetate, 140 mM NaCl, 0.007% PS 80
3. pH 5.0, 10 mM sodium acetate, 140 mM NaCl, 0.007% PS 80
4. pH 6.2, 10 mM sodium phosphate, 140 mM NaCl, 0.007% PS 80

The samples for each formulation were sterile filtered using 0.2 microm PES Syringe filter prior to vialing. The samples are stored in Type I glass Vial with Teflon coated stopper with a fill volume of 0.4 ml. The samples were stored at 4° C. and 25° C. at 60% relative humanity, and 40° C. at 75% relative humanity. The samples were analyzed by a number of analytical methods including SEC-HPLC, RP-HPLC, CZE and cell-based biological activity. An example of the stability analytical data is shown in FIG. 10. The results showed that the formulation at pH 6.2 had the best stability at 25° C. for 6 months, which was followed by the formulation at pH 5.0. It was determined that the formulation samples at pH 4.0 and 4.5 did not appear to be as stable at 25° C. for 6 months.

### Example 8

### Formulation Development—Excipient Screening

Based on the results from the 3 months pH Screening Study, eight formulations as listed below is designed for the excipient screening study.
1. pH 5.2, 20 mM sodium acetate, 140 mM NaCL, 10 mM L-Met, 0.005% PS 80
2. pH 5.5, 20 mM sodium acetate, 5% sucrose, 10 mM L-Met, 0.005% PS 80
3. pH 5.5, 20 mM sodium acetate, 5% sorbitol, 10 mM L-Met, 0.005% PS 80
4. pH 5.5, 20 mM sodium acetate, 140 mM NaCl, 10 mM L-Met, 0.005% PS 80
5. pH 5.2, 20 mM sodium acetate, 140 mM NaCl, 0.005% PS 80
6. pH 5.2, 20 mM sodium acetate, 5% sucrose, 0.005% PS 80
7. pH 5.2, 20 mM sodium acetate, 5% sorbitol, 0.005% PS 80
8. pH 6.2, 20mM sodium Phosphate, 140mM NaCl, 0.005% PS 80

The samples for each formulation were sterile filtered using 0.2 □m PES Syringe filter prior to vialing. The samples are stored in Type I glass Vial with Teflon coated stopper with a fill volume of 0.4 ml. The samples were stored at 4° C. and 25° C. at 60% relative humanity, and 40° C. at 75% relative humanity. The samples were analyzed by a number of analytical methods including SEC-HPLC, RP-HPLC, CZE and cell-based biological activity. An example of the stability data is shown in FIG. 11. Surprisingly, the results showed that formulations containing 140 mM NaCl (#4, #5 and #8) all had acceptable stability after 3 months at 25° C. when detected with CZE. Other analytical results also showed that the stability of formulation #8 had acceptable stability after 3 months at 25° C. (data not shown).

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

```
Sequences
SEQ ID NO: 1
                10 20 30 40
APPRLICDSR VLERYILEAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLCK LFRIYSNFLR GKLTLYTGEA
CRRGDR
SEQ ID NO: 2
                10 20 30 40
APPRLICDSR VLERYILGAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLCK LFRIYSNFLR GKLTLYTGEA
CRRGDR
SEQ ID NO: 3
                10 20 30 40
APPRLICDSR VLERYILEAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLCK LFRIYSNFLR GKLTLYTGEA
CRRGD
SEQ ID NO: 4
                10 20 30 40
APPRLICDSR VLERYILGAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLCK LFRIYSNFLR GKLTLYTGEA
CRRGD
SEQ ID NO: 5
                10 20 30 40
APPRLICDSR VLERYILEAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLRK LFRIYSNFLR GKLTLYTGEA
CRRGDR
SEQ ID NO: 6
                10 20 30 40
APPRLICDSR VLERYILEAR EAENVTMGCN ETCSFSENIT
                50 60 70 80
VPDTKVNFYT WKRMDVGQQA VEVWQGLALL SEAILRGQAL
                90 100 110 120
LANSSQVNET LQLHVDKAVS SLRSLTSLLR ALGAQKEATS
              130 140 150 160
LPEATSAAPL RTFTVDTLSK LFRIYSNFLR GKLTLYTGEA
CRRGDR
DNA Sequence of ASKB626 (Note: The underlined
sequence denotes the signal peptide.)
SEQ ID NO: 7
ATGGGTTCCTGTGAATGCCCTGCCCTCCTCCTCCTGCTGTCCCTGTTGTT
GCTCCCCCTCGGACTCCCGGTCCTGGGCGCGCCCCCAAGACTGATCTGC
GATTCACGCGTGCTGGAGCGGTACATTCTTGAGGCTCGGGAAGCCGAGAA
CGTGACCATGGGTTGTAACGAGACTTGCTCGTTCTCCGAAAACATTACCG
TGCCGGACACCAAGGTCAACTTCTACACCTGGAAACGGATGGACGTGGGA
CAGCAAGCCGTGGAAGTGTGGCAGGGGCTTGCCCTGCTGTCCGAGGCCAT
CCTGCGCGGCCAGGCCCTGCTGGCCAACTCAAGCCAGGTCAACGAGACTC
TGCAACTTCACGTGGATAAGGCCGTGTCGAGCCTGAGGAGCCTCACCTCG
CTCCTGCGGGCACTGGGAGCCCAGAAGGAAGCCACTTCCCTGCCTGAAGC
AACATCCGCTGCGCCGCTGAGGACCTTTACTGTGGACACGCTGTGCAAGC
TGTTCCGCATCTACTCCAATTTCCTGCGGGGGAAGCTGACCTTGTATACC
GGAGAAGCGTGCCGCAGAGGCGACAGATAG
```

1. A pharmaceutical composition or stable sterile aqueous formulation used to treat anemia in cats or dogs, wherein the pharmaceutical composition or stable sterile aqueous formulation comprises a recombinant feline EPO analog molecule that has an amino acid sequence with at least 95% identity to SEQ ID NO: 1; and wherein at least 95% of the recombinant feline EPO analog molecules have at least five (5) N-glycans per molecule, and further wherein, at least 85% of the recombinant feline EPO analog molecules comprise at least one (1) O-glycans per molecule, and wherein at least 10% of the recombinant feline EPO analog molecules have at least two (2) O-glycans per molecule.

2. The pharmaceutical composition or stable aqueous formulation of claim 1, wherein the recombinant feline EPO analog comprises an amino acid sequence selected from SEQ ID NOS: 1-6.

3. The pharmaceutical composition or stable aqueous sterile formulation of claim 1, wherein the pharmaceutical composition or stable aqueous sterile formulation comprises a protein concentration of 0.005-0.10 mg/ml, and a polysorbate-20 or polysorbate-80 concentration of 0.02-0.2 mg/ml; and further wherein said formulation has a pH of 5.0-6.5.

4. The pharmaceutical composition or stable aqueous sterile formulation of claim 1, wherein the pharmaceutical composition or stable aqueous sterile formulation comprises a protein concentration of 0.01-0.05 mg/ml, a polysorbate-80 concentration of 0.025-0.1 mg/ml, phosphate at a concentration of 15-25 mM and sodium chloride at a concentration of 5-10 mg/ml; and wherein the formulation has a pH of 5.9-6.5.

5. The pharmaceutical composition or stable aqueous sterile formulation of claim 1, wherein the pharmaceutical composition or stable aqueous sterile formulation comprises a protein concentration of about 0.025 mg/m I, a polysorbate-80 concentration of approximately 0.05 mg/ml, sodium phosphate monobasic monohydrate at approximately 2.12 mg/ml, sodium phosphate dibasic anhydrous at a concentration of approximately 0.66 mg/ml, and sodium chloride at a concentration of approximately 8.18 mg/ml; and wherein said formulation has a pH at 6.2±0.2.

6. The pharmaceutical composition or stable aqueous sterile formulation of claim 3, wherein the pharmaceutical composition or stable aqueous sterile formulation comprises a protein concentration of about 0.025 mg/ml, polysorbate-80 concentration of approximately 0.05 mg/ml, acetate at 10-20 mM, and sodium chloride at 5-10 mg/ml; and a pH of 5.0-5.8.

7. The pharmaceutical composition or stable aqueous sterile formulation of claim 3, wherein the recombinant feline EPO analog molecules have more negative charges than Darbepoetin alpha, and wherein at least one of the three most abundant bands of the recombinant feline EPO analog molecules runs lower than the lowest band of the four major bands of Darbepoetin alpha as analyzed IEF analysis.

8. The pharmaceutical composition or stable aqueous sterile formulation of claim 3, wherein the recombinant feline EPO analog molecules on average have more negative charges than Darbepoetin alpha, and wherein none of the three most abundant bands of the recombinant feline EPO analog molecules runs higher than the second highest band of the four major bands of Darbepoetin alpha as analyzed by IEF analysis.

9. The pharmaceutical composition or stable aqueous sterile formulation of claim 3, wherein the recombinant feline EPO analog molecules on average have more negative charges than Darbepoetin alpha, and wherein none of the three most abundant peaks of the recombinant feline EPO analog molecules eluted earlier than the second earliest eluted peak of the three most abundant peaks of Darbepoetin alpha as analyzed by CZE analysis.

10. The pharmaceutical composition or stable aqueous sterile formulation of claim 3, wherein the recombinant feline EPO analog molecules on average have more negative charges than Darbepoetin alpha, and wherein at least two of the three most abundant peaks of the recombinant feline EPO analog molecules eluted later than the last eluted peak of the three most abundant peaks of Darbepoetin alpha as analyzed by CZE analysis.

11. The pharmaceutical composition or stable aqueous sterile formulation of claim 3, wherein the recombinant feline EPO analog is glycosylated; and wherein at least 95% of the recombinant feline EPO analog molecules contain at least five N-glycans per molecule, and wherein, at least 85% of said EPO analog molecules comprise at least one (1) O-glycans per molecule, and further wherein at least 20% of the feline EPO analog molecules contain two or more O-linked glycans per molecule.

12. The pharmaceutical composition or stable aqueous sterile formulation of claim 3, wherein the recombinant feline EPO analog is glycosylated; and wherein at least 95% of the recombinant feline EPO analog molecules contain at least five N-glycans per molecule, and further wherein at least 85% of said EPO analog molecules comprise at least one (1) O-glycans per molecule, and wherein at least 30% of the feline EPO analog molecules contain two or more O-linked glycans per molecule.

13. The pharmaceutical composition or stable aqueous sterile formulation of claim 3, wherein the recombinant feline EPO analog is glycosylated; and wherein at least 95% of the recombinant feline EPO analog molecules contain at least five N-glycans per molecule, and further wherein at least 85% of said EPO analog molecules comprise at least one (1) O-glycans per molecule, and wherein at least 40% of the feline EPO analog molecules contain two or more O-linked glycans per molecule.

14. The pharmaceutical composition or stable aqueous sterile formulation of claim 3, wherein the recombinant feline EPO analog is glycosylated; and wherein at least 95% of the feline EPO analog polypeptide molecules in the pharmaceutical composition or stable aqueous sterile formulation have up to 18 or more sialic acids in each recombinant feline EPO analog molecule.

15. The pharmaceutical composition or stable aqueous sterile formulation of claim 3, wherein the recombinant feline EPO analog is glycosylated; and wherein at least 95% of the feline EPO analog molecules in the pharmaceutical composition or stable aqueous sterile formulation have up to 19 or more sialic acids in each recombinant feline EPO analog molecule.

16. The pharmaceutical composition or stable aqueous sterile formulation of claim 3, wherein the recombinant feline EPO analog molecules are glycosylated; and wherein at least 98% of the feline EPO analog polypeptide molecules in the pharmaceutical composition or stable aqueous sterile formulation have up to 19 or more sialic acids in each recombinant feline EPO analog molecule.

17. The pharmaceutical composition or stable aqueous sterile formulation of claim 3, wherein the recombinant feline EPO analog is glycosylated; and wherein at least 90% of the recombinant feline EPO analog molecules in the pharmaceutical composition or stable aqueous sterile formulation comprise up to 20 or more sialic acids in each recombinant feline EPO analog molecule.

18. The pharmaceutical composition or stable aqueous sterile formulation of claim 3, wherein the level of aggregated protein is less than 2% as analyzed by a SEC-HPLC method.

19. The pharmaceutical composition or stable aqueous sterile formulation of claim 3, further comprising 2-20 mM methionine.

20. (canceled)

21. A method for treating anemia in a subject, wherein said subject comprises a cat or a dog, said method comprising administering to a subject in need a pharmaceutical composition of claim 1.

22.-28 (canceled)


SEQUENCE LISTING (7 sequences)

SEQ ID NO: 1 - protein, 166 aa, artificial
  recombinant feline erythropoietin (EPO) analog
APPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGDR

SEQ ID NO: 2 - protein, 166 aa, artificial
  recombinant feline erythropoietin (EPO) analog
APPRLICDSRVLERYILGAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGDR

SEQ ID NO: 3 - protein, 165 aa, artificial
  recombinant feline erythropoietin (EPO) analog
APPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGD

SEQ ID NO: 4 - protein, 165 aa, artificial
  recombinant feline erythropoietin (EPO) analog
APPRLICDSRVLERYILGAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLCKLFRIYSNFLRGKLTLYTGEACRRGD

SEQ ID NO: 5 - protein, 166 aa, artificial
  recombinant feline erythropoietin (EPO) analog
APPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLRKLFRIYSNFLRGKLTLYTGEACRRGDR

SEQ ID NO: 6 - protein, 166 aa, artificial
  recombinant feline erythropoietin (EPO) analog
APPRLICDSRVLERYILEAREAENVTMGCNETCSFSENITVPDTKVNFYTWKRMDVGQQAVEVWQGLALLSEAILRGQALLANSSQVNETLQLHVDKAVSSLRSLTSLLRALGAQKEATSLPEATSAAPLRTFTVDTLSKLFRIYSNFLRGKLTLYTGEACRRGDR

SEQ ID NO: 7 - DNA, 579 bp, artificial
  DNA Sequence of ASKB626 (first 77 nucleotides encode the signal peptide)
atgggttcctgtgaatgccctgccctcctcctcctgctgtccctgttgttgctccccctcggactcccggtcctgggcgcgcccccaagactgatctgcgattcacgcgtgctggagcggtacattcttgaggctcgggaagccgagaacgtgaccatgggttgtaacgagacttgctcgttctccgaaaacattaccgtgccggacaccaaggtcaacttctacacctggaaacggatggacgtgggacagcaagccgtggaagtgtggcaggggcttgccctgctgtccgaggccatcctgcgcggccaggccctgctggccaactcaagccaggtcaacgagactctgcaacttcacgtggataaggccgtgtcgagcctgaggagcctcacctcgctcctgcgggcactgggagcccagaaggaagccacttccctgcctgaagcaacatccgctgcgccgctgaggacctttactgtggacacgctgtgcaagctgttccgcatctactccaatttcctgcgggggaagctgaccttgtataccggagaagcgtgccgcagaggcgacagatag